(19) United States
(12) Patent Application Publication
(10) Pub. No.: US 2008/0312543 A1
(43) Pub. Date: Dec. 18, 2008

(54) MEASUREMENT OF PULMONARY HYPERTENSION FROM WITHIN THE AIRWAYS

(75) Inventors: Michael D. LAUFER, Menlo Park, CA (US); Edmund J. ROSCHAK, Mission Viejo, CA (US)

Correspondence Address: Broncus Technologies, Inc. c/o;Levine Bagade Han LLP, 2483 E. Bayshore Road Suite 100, Palo Alto, CA, 94303, US

(73) Assignee: BRONCUS TECHNOLOGIES, INC., Mountain View, CA (US)

(21) Appl. No.: 12/141,733

(22) Filed: Jun. 18, 2008

Related U.S. Application Data
(60) Provisional application No. 60/944,730, filed on Jun. 18, 2007.

(51) Int. Cl. A61B 5/02

(52) U.S. Cl. 600/486

(57) ABSTRACT

This is directed to methods and devices suited for airway based measurements of pressure in a pulmonary artery. A device is advanced into an airway and in the vicinity of the pulmonary artery. Physical properties of the pulmonary artery are observed through the airway wall using one or more minimally invasive modalities. In a variation, a bronchial balloon catheter measures pressure of the pulmonary artery.

### CROSS-REFERENCE TO RELATED APPLICATIONS

This application claims priority to U.S. Provisional Application No. 60/944,730, filed Jun. 18, 2007, which is hereby incorporated by reference in its entirety.

### BACKGROUND OF THE INVENTION

Pulmonary arterial hypertension (PH) is a continuous abnormally high mean blood pressure in the pulmonary artery or arteries of the lungs. In a healthy individual, the average resting blood pressure in these arteries is about 14 mmHg. In individuals suffering from PH, the average resting blood pressure in these arteries is usually greater than 20 or 25 mmHg.

Pulmonary arteries carry oxygen deficient blood from the right heart ventricle to the small arteries in the lungs where the blood becomes oxygenated. The abnormally high pressure is associated with changes in the small blood vessels in the lungs as well as with abnormalities in the heart, sometimes referred to as cor pulmonale. The changes in these small blood vessels results in an increased resistance to blood flowing through the vessels, and a further increase in the pressure required to maintain the flow.

The increased resistance in the blood vessels increases the amount of effort required by the heart's right ventricle to move adequate amounts of blood through the lungs. As a result, the right ventricle must pump harder. This increased workload eventually causes the right side of the heart to become enlarged. Eventually, this condition can lead to heart failure.

Typically, there are two types of PH: Primary PH and secondary PH. Primary PH is believed to be inherited or may even occur for no known reason. Secondary PH is believed to occur because of another condition. Such conditions may include chronic heart or lung disease, blood clots in the lungs, or other diseases.

PH is a common complication of chronic obstructive pulmonary disease (COPD). It is sometimes called cor pulmonale, as it is heart failure caused by lung dysfunction. The cause of PH in COPD patients is generally assumed to be hypoxic pulmonary vasoconstriction as a result of the lack of oxygen or constriction due to acidemia from an inability to exhale carbon dioxide. In those COPD patients suffering from moderate to severe PH, the patient not only suffers from hyperinflated lungs and a lack of oxygen and acidemia, but the patient may also begin to suffer from complications associated with right-sided heart failure. Other theories include the fact that hyperinflated lungs require blood to flow at greater pressure in order to perfuse the hyperinflated segments. In addition, patients with hyperinflation are unable to sufficiently decrease the intrathoracic pressure. This further compromises their heart's ability to pump, as less blood is returned to the heart, resulting in insufficient priming, making the heart function inefficiently. Physiologists refer to this phenomenon as being on the left side of the Frank-Starling curve.

Those afflicted with COPD face disabilities due to the limited pulmonary function and cardiovascular function. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease. If the pulmonary component can be reversed, such as by lung transplantation, lung volume reduction surgery, or airway bypass decompression, some of these cardiovascular effects can be sometimes improved. Additionally, medications have been developed to reduce pulmonary hypertension, such as sildenafil citrate.

Indirect tests for PH include electrocardiography, chest x-ray, computed tomography (CT or CAT scan), Magnetic resonance imaging (MRI), tests to measure cardiac output and Doppler echocardiography, but right-heart catheterization is the only direct test for PH and considered by most to be the Gold Standard.

Problems arise in patients with COPD since the tests for PH require specialized equipment or invasive catheterization techniques that are generally outside the scope of practice for pulmonologists, and are generally practiced only by cardiologists or Intensivists in an Intensive Care setting. These tests are currently not done in an outpatient or clinic setting. Accordingly, physicians treating or examining a COPD patient in a clinic or bronchoscopy suite using bronchoscopic techniques do not have the tools to monitor potential PH in the patient without subjecting the patient to additional tests. If the physician were able to observe the degree of PH during a bronchoscopic examination or treatment, the physician would be able to treat the patient's PH as well as monitor (or treat) the patient's COPD. Furthermore, they would, for the first time, be able to institute treatment for PH and monitor its effectiveness, instituting changes in dosage or medication or instituting other techniques with feedback from these measurements.

Accordingly, a need remains for a physician to be able to measure pressure pulmonary arteries from within the airways.

### BRIEF SUMMARY OF THE INVENTION

The invention includes a method for observing, or calculating from observations, a pressure, or set of pressures, in a pulmonary artery, the method comprising identification of the target pulmonary vessel or measurement location, advancing an expandable device within an airway of a patient, pressurizing the expandable device against an airway wall to compress a targeted pulmonary artery adjacent to the airway wall in a sufficient amount to interfere with the flow of blood in the pulmonary artery, identifying the interference with flow in the targeted vessel while observing a pressure of the expandable device, and correlating the interference of the flow of blood in the pulmonary artery with the pressure in the expandable device to determine the pressure in the pulmonary artery.

The above method may include pressurizing the expandable device to stop the flow of blood in the pulmonary artery while observing the pressure of the expandable device while observing the pressure when blood flow stops.

In another variation, the method may further comprise subsequently depressurizing the expandable device to re-establish blood flow, while observing the pressure of the expandable device comprises observing the pressure when blood flow is re-established, and continuing to monitor the pressure and flow until the interference with flow is completely removed.

Another variation of the invention includes observing a pressure in a pulmonary artery by advancing a first device into an airway, measuring a first flow rate of blood flow in the pulmonary artery with the first device, pressurizing an expandable member within an airway to interfere with the blood flow in the pulmonary artery, measuring a second flow rate of blood flow in the pulmonary artery; and comparing the first flow rate to the second flow rate to determine the pressure in the pulmonary artery. Without being bound to theory, this follows from the principle that the pressure applied extrinsically by the balloon increases the flow rate (and increases the resistance of the vessel) by the measured amount. Therefore, a line can be constructed and extrapolated for the pressure at which flow would stop. This is representative of the systolic pressure in the vessel being compressed.

Further, the ratio of pressure over flow at the first condition is equal to the ratio of pressure to flow in the second condition. If the amount of force required to compress the vessel in order to increase the flow rate at the second condition is known, this pressure of compression plus the pressure in the vessel when compressed is equal to the pressure initially in the vessel. The flow rate determines what proportion of the vessel is compressed, so long as the vessel is not occluded. That proportion multiplied by the pressure in the compressing element equals the pressure in the vessel. If the vessel can be completely occluded, of course, the pressure of occlusion is equal to the pressure in the vessel.

In another variation, a method for observing a pressure in a pulmonary artery includes advancing a device into an airway of a lung, locating the pulmonary artery along a surface of the airway, assuring that it is deoxygenated blood as well as pulsatile arterial blood (consistent with a pulmonary artery and not a bronchial artery), inserting a pressure measuring device through the airway wall directly into the pulmonary artery, measuring the pressure of the pulmonary artery with the pressure measuring device, and removing the pressure measuring device from the pulmonary artery, with the capacity of applying direct pressure to the area of insertion to limit bleeding once the inserted member is removed.

Yet another variation of the invention includes locating the pulmonary artery along a surface of the airway, placing a motion sensing device, such as an accelerometer, against the surface of the airway near a location of the pulmonary artery, measuring an attribute of movement of the airway wall resulting from the movement, flow or change in flow or movement of blood within the pulmonary artery, determining a radius of the pulmonary artery, and calculating an increase in pressure of the pulmonary artery using the amount of movement of the airway wall and the radius of the pulmonary artery. For example, on placing an accelerometer against the airway wall and adjacent to the pulmonary artery the medical practitioner is able to determine the strain in the arterial wall related to the acceleration of blood. The strain in the arterial wall correlates to the pressure in and size of the artery. If the medical practitioner determines the size or radius of the arterial wall, then the practitioner can calculate the pressure.

Another variation of invention comprises placing a device against the airway wall. The device comprises a known mass and an accelerometer. The device and accelerometer are moved by the pulse of blood through the pulmonary artery. Acceleration is measured using the accelerometer. The mass is then changed by a known amount to another mass. The mass may be changed by, for instance, injecting saline into the device. Next, a second acceleration is then measured. The various measurements of mass and accelerations allow the medical practitioner to calculate force or pressure in the vessel. Force is indicative of the pulmonary pressure in the pulmonary artery and is therefore a useful measurement. The force is also correlated to pressure by the area of the vessel of the patient. Observing force measurements and changes over time also indicates a trend or change in pulmonary pressure.

The method may also include locating the pulmonary artery along a surface of the airway until it is in the vicinity of the artery, advancing a device into an airway of a lung, where the device is configured to measure the Doppler flow velocity pattern in the pulmonary artery, recording flow patterns or waveforms over a period of time and comparing these waveforms against waveforms characteristic of normal and a number of high pressures in the pulmonary artery.

Another variation of the invention includes carrying out any one or more of the techniques described herein subsequent to a treatment or therapy, and or carrying out the techniques periodically. The method may further include comparing the measured information to previously obtained information and identifying a change, trend, or increase in pulmonary pressure or disease. Initial testing may also be calibrated with a base line value such as a pressure determined using a right heart catheterization.

### BRIEF DESCRIPTION THE DRAWINGS

FIG. 1 illustrates an example of placing an expandable device into an airway.

FIG. 2 illustrates expansion of the device of FIG. 1 to stop or impede flow in a pulmonary artery.

FIG. 3a illustrates an example of inserting a pressure sensor or transducer directly into a pulmonary artery through an airway wall.

FIG. 3b illustrates an example of a tamponade device comprising a tapered surface.

FIG. 3c illustrates an example of a tamponade device extending from the needle device.

FIG. 3d illustrates an example of a tamponade device in an application to close a wound on a blood vessel.

FIG. 4 illustrates an example of a device that measures flow in a pulmonary artery so that the measurements can be compared to characteristic or base information for an assessment of the pressure in the pulmonary artery.

FIG. 5 is an illustration of a chart correlating pulmonary pressure and lag time.

### DETAILED DESCRIPTION

Described herein are devices (and methods) for measuring pulmonary hypertension (PH) or simply measuring pressure within the pulmonary arteries of a patient from devices advanced within the airways. As noted above, the methods and devices allow a physician to observe conditions of PH by placing a bronchoscopic or other device within the airways of the patient. These methods and devices have particular applicability to monitoring the PH of a COPD patient. However, the methods and devices are not limited to use in COPD patients. Additionally, variations of the invention include less or non-invasive techniques including obtaining measurements from outside the body such as through the chest wall as will be described further herein.

In various embodiments of the invention the location of a blood vessel is found by advancing a Doppler device (or any other device capable of locating a blood vessel beyond a wall of the airway.) The Doppler device may comprise a catheter and an ultrasonic transducer located towards the distal end or distal portion of the catheter. The ultrasound transducer sends and receives signals to and from the tissues. When the signals are directed towards a blood vessel transporting blood, a difference in the afferent and efferent frequencies can be detected as a Doppler shift. A signal processing unit connected to the Doppler device processes the signals and provides feedback to the physician as to whether a vessel is in the vicinity of the catheter distal end/distal portion. An example of an ultrasonic Doppler device to sense the presence of a blood vessels is described in U.S. Pat. No. 7,022,088 to Keast et al. which is hereby incorporated by reference in its entirety.

Bronchial Catheter with Pressure-Sensing Balloon

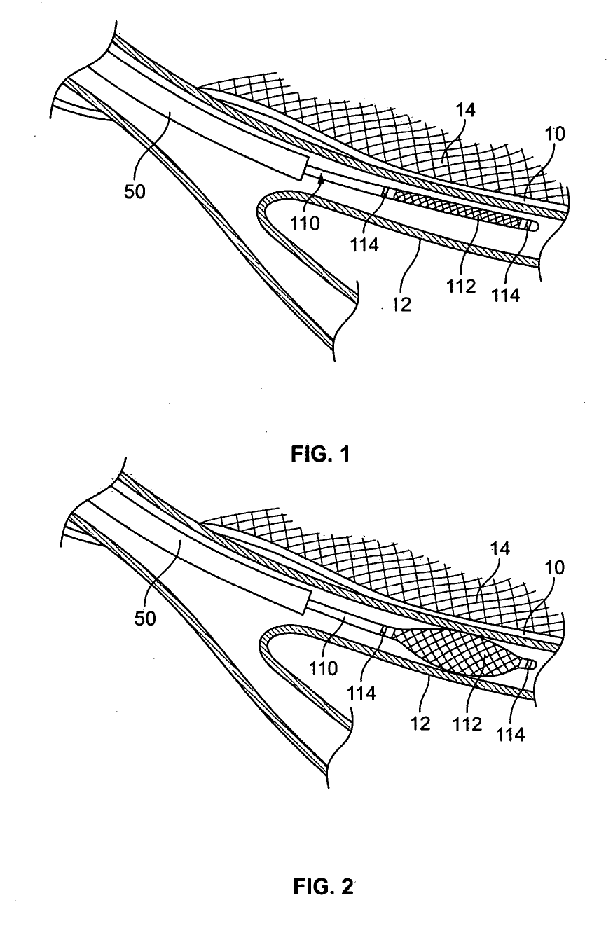

As shown in FIG. 1, once the location of a pulmonary artery 10 is identified, the medical practitioner advances an expandable device 110 within the airway to the region of the blood vessel 10. The device 110 may optionally be advanced using an access device 50 such as a catheter or bronchoscope. The expandable device 110 may comprise a distensible or non-distensible balloon 112. Additional variations of the device can include non-balloon expandable devices (e.g., such as expandable basket type devices or other mechanical type expansion devices). In any case, it must be possible to observe and measure the pressure that the expandable member exerts on the airway as it expands.

Next, as shown in FIG. 2, the balloon or expandable member 112 is pressurized. Upon expansion, the balloon 112 expands the airway 12 to interfere with flow of blood through the pulmonary artery 10. It is believed that tissue structures 14 adjacent to the pulmonary artery 10 will provide a resisting surface to compress the artery. The region along the pulmonary artery in the vicinity of the Ligamentum Arteriosum is an example of a desired location. The Ligamentum Arteriosum is an area of increased tissue fixation because this connective tissue joins the pulmonary artery to the aortic arch.

In order to observe the pressure within the pulmonary artery 10 the balloon can either fully cease flow within the artery or partially interfere with the flow of blood in the artery. Also, the angle of incidence of the sound waves should not change while the vessel is being compressed as that could decrease the accuracy of the measurement.

In those cases where blood flow is stopped. The practitioner can observe or record the pressure of the balloon that was sufficient to stop blood flow. Alternatively, or in combination, the practitioner can fully stop blood flow or re-establish blood flow. In either case, correlating the pressure at which the blood flow stops, or at which the blood flow resumes, allows for measurement of the pressure of the pulmonary artery. In such a procedure, it will be important to expand the device in areas where the pressure required to expand the airway (to ultimately compress the vessel) is less than that of the pressure of the pulmonary artery itself. Locations along the airway and between cartilagous rings are desirable locations.

Another variation of the invention applies a pressure to the pulmonary artery such that the pulmonary artery is prevented from expanding but not completely occluded. The pressure to the pulmonary artery is directionally applied from the airway using, for example, a bronchial balloon catheter. The bronchial balloon catheter prevents displacement of the airway arising from the pulmonary arterial pulse. The pressure is recorded at the point that pulmonary arterial pulse pressure is about equal to the force arising from the interventional instrument (i.e., the bronchial balloon catheter, or other device that may apply a known force). In this manner, the pulmonary arterial pressure is measured.

Although a bronchial balloon catheter is described above, the invention is not so limited. Other force generation instruments may be used to prevent the airway wall from moving due to the pulmonary arterial pulse. For example, a known weight placed over the pulmonary artery provides a known pressure due to gravity on the pulmonary artery. The force and corresponding pressure could be calculated. The weight required to prevent the airway wall from moving is recorded. Hence, the pressure may be measured.

In another variation, a controller is adapted to control inflation of the bronchoscopic balloon or inflatable member so as to physically prevent the expansion of the pulmonary artery during a pressure pulse. In this variation, the balloon is inflated at the instant that the pulmonary artery is expanding corresponding to the heart contracting. The pressure of the balloon at this point corresponds to the systolic pressure in the artery. The controller shall contain the software, connections, and hardware to control inflation of the balloon at the correct instant, and to measure the pressure of the balloon at this time.

Although the above controller is described as automatic, it is also within the scope of the present invention to provide a manual inflation device to control the balloon. Examples of manual devices include syringes, or columns of fluid. Balloon inflation is preferably observed with a bronchoscope, or another visual or audio instrument so as to ensure that the balloon is inflated at the correct instant.

In the event a fluid is used to provide the working force or pressure, and the fluid pressure is not automatically controlled by a control system as discussed above, it may be necessary to build a column of fluid outside of the patient that creates sufficient force. To this end, the column of fluid preferably is positioned above the patient. Indeed, the pulmonary pressure may be upwards of 25-30 mm Hg. In this instance, the movement of the column meniscus as the pressure changes with each pulse will indicate the pulmonary arterial pressure when properly calibrated.

In some cases it may be difficult to compress the artery 10 from within the airway. Accordingly, another variation of the method includes making a hole in the airway wall, advancing the balloon through the hole, placing the balloon against the pulmonary artery (so the balloon is on one side of the artery and the airway is on the other) and inflating the balloon to compress the pulmonary artery. A variation includes inserting a needle through the airway wall to access the space adjacent a vessel through which or over which a device is placed into that same space to stop or interfere with blood flow. Such a variation can use a pressurized balloon advanced through the needle to stop or cease blood flow. Additionally, advancing a sensor device through the airways, and making a hole in the airway in the vicinity of the pulmonary artery, advancing the sensor device through the hole in the airway towards the pulmonary artery from which to obtain pulmonary information or data is an aspect of the invention. The sensing may therefore be performed from within the airway, outside the airway and adjacent the pulmonary artery, and or within the pulmonary artery.

Obviously, it is extremely important that the flow of blood in the pulmonary artery is not stopped or disrupted for such a duration to cause harm to the patient.

The device 110 itself can be equipped with Doppler transducers 114. Alternatively, or in combination, Doppler transducers can be located in the balloon or on a separate device that is advanced to the site. In any case, any standard Doppler technique can be used to identify the flow or cessation of flow of blood within the artery.

In another variation, the method may include advancing a first device into an airway and using the device to measure a first flow rate of blood flow in the pulmonary artery. Examples of devices that may be positioned in the airway and measure blood flow in the pulmonary artery include Doppler devices. The flowrate is determined using a pulsed (gated) Doppler technique. This technique is described in various references and patents such as in U.S. Pat. No. 4,327,739 which is hereby incorporated by reference in its entirety.

Next, a balloon or other member is pressurized as an expandable member within an airway to interfere with the blood flow in the pulmonary artery. Such interference can include partially or completely occluding the airway while also partially or completely occluding the adjacent vessel. Next, the flow measurement device takes another measurement of the flow rate of the partially occluded vessel. Accordingly, the first and second flow rates with the degree of occlusion of the blood vessel should allow for calculation of the pressure within the pulmonary artery. Although such a measurement is an indirect measurement of the pressure within the pulmonary artery, it is equally useful.

Bronchial Catheter with Vessel-Penetrating Needle and Pressure Transducer

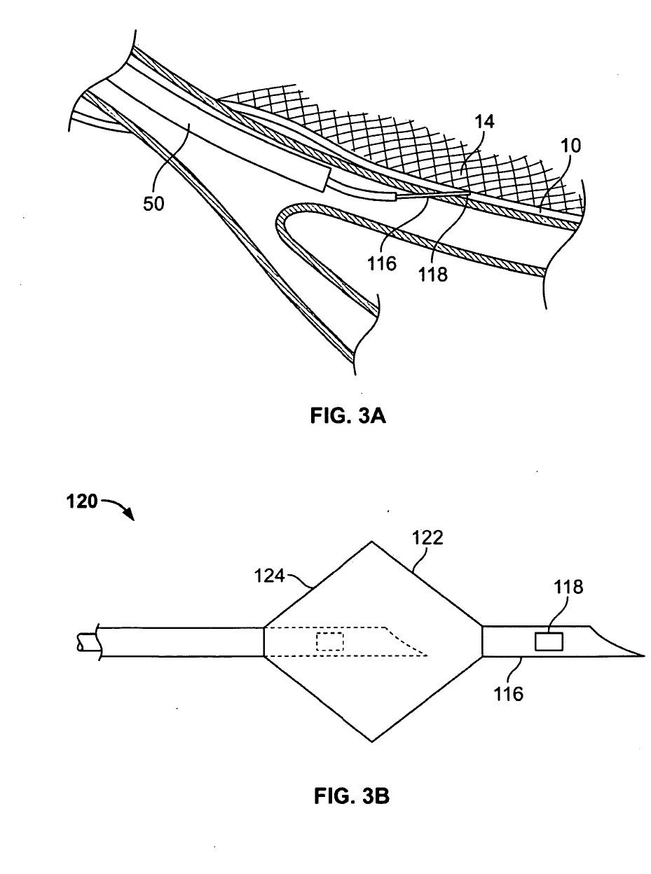

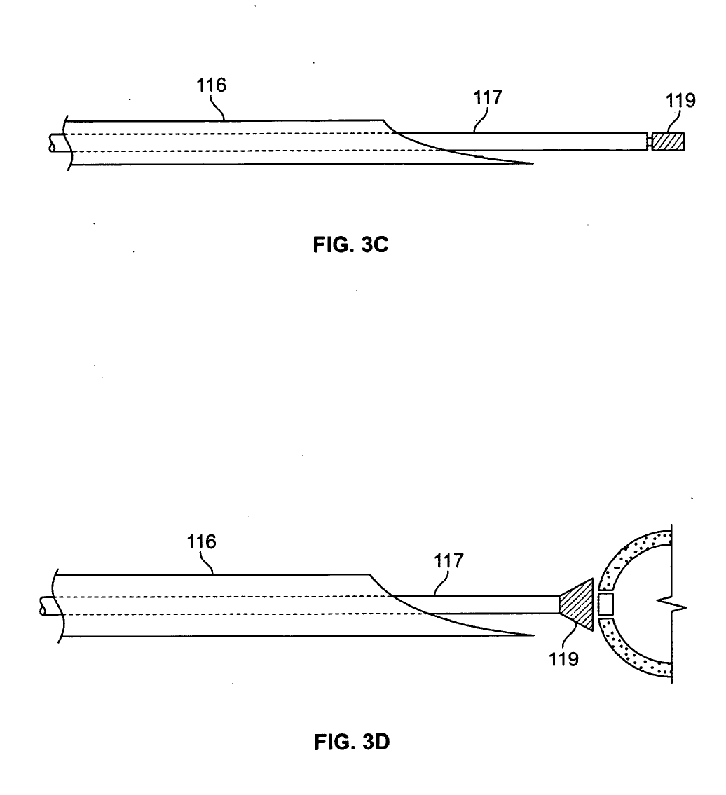

FIG. 3a illustrates another variation of a method according to the present invention. As noted above, the medical practitioner locates the pulmonary artery along a surface of the airway. Once the location is determined, the medical practitioner advances a needle-type device 116 through an airway of a lung to the surface where the artery is identified. The needle-type device 114 includes a pressure transducer 118 or is equipped to measure pressure in a tip portion or distal end of the needle member. The medical practitioner then inserts the pressure measuring needle through the airway wall directly into the pulmonary artery. Naturally, the needle-type device may simply be a cannula or other elongate member that is able to access the artery through the airway wall. Once inside the artery, the medical practitioner measures the pressure of the pulmonary artery with the pressure measuring device. Such a method yields an equivalent measurement of pulmonary arterial pressure as the right heart catheterization technique discussed above. Naturally, once the medical practitioner obtains the pressure measurement the needle member 116 is withdrawn from the artery and airway wall. It may be necessary to apply a sealant or pressure to seal the opening created by the device 116 to prevent excessive post procedure bleeding. A bronchial balloon may be used to apply external pressure to the opening of the vessel.

FIG. 3b illustrates a tamponade device 120 comprising a tapered portion 122. The tamponade may be part of the needle type device 116, or a separate catheter. The tamponade device can prevent leakage of blood while the working end or pressure transducer of the needle device is within the blood vessel. The tamponade device may move relative to the needle tip. In an application, the needle device is inserted into the blood vessel. Once inserted the tamponade is expanded to prevent leakage of blood through the surgically created opening. After the pressure has been observed, the tamponade is reduced and the needle device removed. External pressure may then be applied to facilitate sealing the wound.

In another variation, the tamponade is used following withdrawal of the needle from the blood vessel. Once the needle is withdrawn from the blood vessel 10, the needle member 116 is retracted within the tapered portion 122. The tapered portion 122 is urged against or into the puncture hole in the blood vessel wall. In this manner external pressure is applied to the surgical hole. The tapered portion 122 is held against the vessel hole until the wound is sealed. Examples of time to hold the tamponade in place include about 3 minutes or more. However, the hole or wound may seal in less time and the hold time may be adjusted accordingly.

In the variation shown in FIG. 3b, the needle does not allow blood therethrough or to otherwise freely escape from the vessel. In order for the tamponade to operate effectively, the blood must be contained to some degree. The needle thus preferably comprises a plug, block, or clotting surface to prevent fluid from flowing therethrough. Alternatively, the needle may be solid.

The shape of the tapered portion may vary greatly. The taper is preferably adjustable and is expandable from a low profile configuration to an expanded configuration. The taper may be expanded using various mechanisms such as, for example, fluid pressure, elasticity, pivoting and rotation members, a bellows or accordion design, and/or other techniques. In an alternative embodiment, the tamponade is not expandable and comprises a continuous taper along the catheter shaft. In another variation, the needle device is completely removed and the tamponade is applied to the puncture hole to treat the wound.

FIG. 3b also illustrates a reverse tapered portion 124. The reverse tapered portion facilitates withdrawal of the tamponade through the lumen walls, and other openings. In particular, the reverse taper serves to reduce damage to the wall as the device is withdrawn. The reverse taper may be expandable and share other characteristics with the forward tapered portion 122. Although not shown, the instrument may be formed of a plurality of catheters that cooperate together. One catheter may comprise a forward taper, and another catheter may comprise the reverse taper.

Fluid channels or openings may be incorporated into the tapered portion to locally deliver sealants such as a fibrin agent or another clotting agent to seal the puncture. Medicants may also be supplied thought the openings including pressure affecting medicines such as Viagra®.

FIG. 3c illustrates another variation of a tamponade device. The tamponade device shown in FIG. 3c features a shaft 117 that is movable relative to the needle device 116. The shaft 117 may be extended and retracted from within the needle device. The shaft 117 terminates at a distal end which is shown having a clotting material 119.

In an application, and as shown in FIG. 3d, the clotting material 119 is urged against a blood vessel. The clotting material is preferably urged against the outside of the blood vessel to provide external pressure and homeostasis. The clotting material may be soft or hard. The clotting material preferably is expandable. In this manner, the clotting material may form a proper size tamponade to cover the hole in the blood vessel. There are a variety of clotting materials that may be positioned or attached to the shaft distal end. Examples of materials include cellulose foam, quickclot (Zeolite), alumino phosphate, chitozan, cotton gauze, and gelatinous materials including, for example, carboxy methylcellulose. Once the tamponade has been placed in position to seal the wound, and the wound has stopped bleeding, the shaft 117 is retracted within the needle device 116. The blood vessel is thus sealed and removal of needle device may be accomplished.

Bronchial Catheter with Strain Transducer

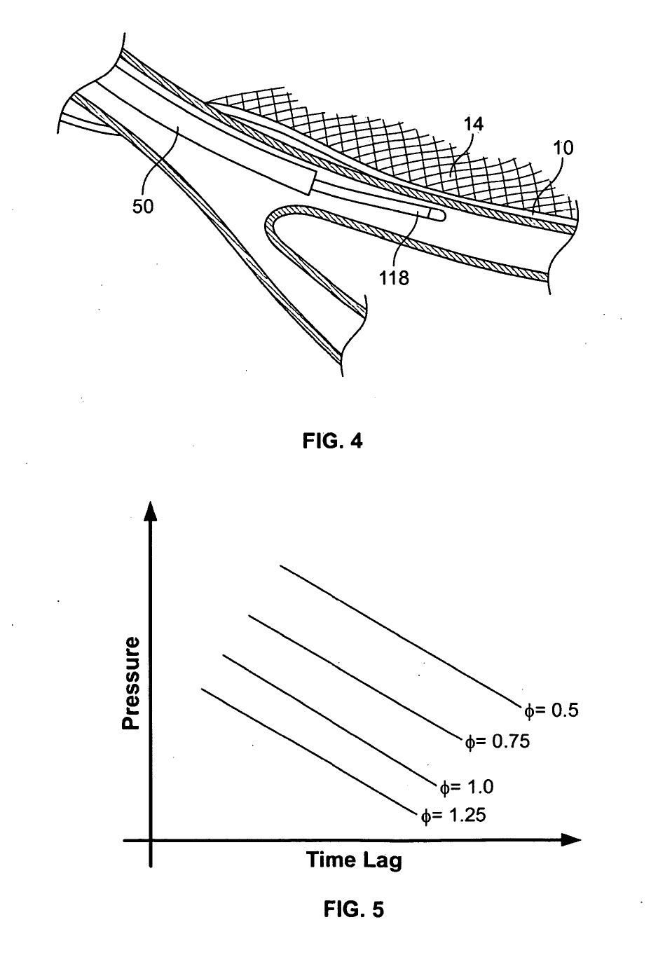

FIG. 4 shows yet another variation of observing a pressure in a pulmonary artery. In this variation the measurement of pressure in the pulmonary artery occurs indirectly by comparing actual measurements of blood flow in the pulmonary artery against various parameters. As shown, a measurement catheter 118 is placed against an area in the airway where the medical practitioner previously determined the existence of a blood vessel. In one variation, the device comprises placing a motion sensing device against the surface of the airway near a location of the pulmonary artery. The motion-sensing device can comprise any transducer based device, a strain gauge, or a device having an accelerometer. In this variation, the device should measure an amount of movement of the airway wall resulting from acceleration of blood within the pulmonary artery. By doing so, the device detects the strain in the artery wall as a result in the increase in pressure that results from the flow of blood as the heart pumps. Prior to this, the medical practitioner shall use known means to determine a diameter or radius of the artery wall. This may be accomplished through any various modes of imaging (e.g., CT scans, Doppler imaging, etc.) Next, the increase in size or strain in the artery is correlated to the pressure increase required to produce such a strain to calculate the pressure within the artery wall.

Another variation of invention comprises placing a device against the airway wall. The device comprises a known mass and an accelerometer. The device and accelerometer are moved by the pulse of blood through the pulmonary artery. Acceleration is measured. The mass is changed by a known amount to another mass. The mass may be changed by, for instance, injecting saline into the device. A second acceleration is then measured. The various measurements of mass, and accelerations allow the medical practitioner to calculate force, or pressure. Force is indicative of the pulmonary pressure in the pulmonary artery and is therefore a useful measurement. Force is correlated to pressure by the area of the vessel of the patient. Indication of the force over time also indicates trend or pulmonary pressure changes.

Another variation involves identifying a location along the airway that is moving with the pulsatile expansion of the pulmonary artery. This variation involves observing or measuring the displacement or movement of the airway wall with an endoscope, bronchoscope, or another viewing technology. The video image, or scan, may be stored and analyzed using, for example, software to estimate the displacement or strain on the tissue. Additionally, the modulus of elasticity of the tissue structures (namely the pulmonary artery and airway wall) may be estimated and consequently, the stress or pressure may be estimated using Hooke's law. This pressure corresponds to the heart contracting, and the systolic pressure.

Bronchial Catheter with Thermistor

Another variation of the invention comprises determining the flowrate through the pulmonary artery. A catheter comprising a thermistor may be advanced to a suitable location along the airway. The thermistor is placed against the wall of the airway at a location adjacent to the pulmonary artery. A bolus of cold fluid (e.g., 10 cc of saline) is injected into the pulmonary artery and the temperature profile downstream of the injection is monitored. The flowrate may be calculated from the temperature profile in combination with the known volume of fluid. Although it is desirable to inject the bolus of fluid near the temperature probe, the invention is not so limited. The bolus may be injected in another location such as into the vessels in the arm. Additionally, if the volume of the right ventricle (such as from an echo) and the flowrate are known, one should be able to calculate pressure since the vessel size is known (which provides resistance).

In another variation, the thermistor is placed into or near the pulmonary artery using a needle which has been manipulated or advanced through the airway as described above.

Waveform Analysis with Bronchial Doppler Catheter

In another variation shown in FIG. 4, the medical practitioner places the device 118 against the airway wall near the pulmonary artery 10, the practitioner obtains waveform information of blood flowing within the artery 10. The device 118 may rely on a Doppler Effect measurement to obtain flow-rate or velocity waveforms. The measurements may occur over a duration of time or over a number of heart beats. The actual waveforms are then compared to known waveform information. For example, the waveforms of healthier individuals can be established as a baseline where the measured flow pattern is then compared to the base waveform to assess whether a flow pattern difference indicates pulmonary hypertension.

Additionally, the angle of incidence of the Doppler is preferably accounted for or known. The angle of incidence may vary and preferably be about 30-45 degrees incidence with the direction of blood flow.

Methods of non-invasively determining pulmonary hypertension by Doppler or other means are discussed in Non-Invasive Evaluation of Pulmonary Hypertension by a Pulsed Doppler technique by A. Kitabatake (Circulation 1983; 68; 302-309). The entirety of which is hereby incorporated by reference. However, these techniques require visualization from outside the body. Moreover, such external imaging of COPD patients is difficult in view of the large amount of air trapped within hyper-inflated lungs. More direct measuring of characteristics of blood pressure and assessing pulmonary hypertension from within an airway under the present invention overcomes these problems. The above referenced methods and procedures may be carried out using various instruments, devices, and systems. In one variation, one or more catheters having a flexibility and size sufficient to advance and navigate through the airways is provided. The catheter includes a Doppler ultrasound sensor. The catheter is connected to a controller that provides electrical stimulus to activate the ultrasound transducer. The controller also comprises a signal processing unit that controls and processes the transmitted and return signals. The controller provides various feedback to the user including, for example, audio or visual feedback.

The Doppler signal processing unit may indicate the presence of flow by observing the Doppler frequency shift. Additionally, localized velocity of the fluid may be determined using, for example, a gated (Pulsed) Doppler ultrasound technique. This may also be used to determine the diameter of the vessel by gating the signal until no flow is seen and then gating it back through the vessel until no flow is again seen. The difference between one “no-flow” region and the next is the diameter of the vessel.

Lag Time Measurement

In another variation, a lag time between a heart contraction and a pressure pulse along the pulmonary artery is measured. Lag time may be obtained relatively easily and frequently over time. The measurements are recorded and compared. A change, trend, or difference in the measurements over time may indicate a change in the underlying pulmonary arterial pressure. This is believed to follow from the principle that an increase in pressure in the pulmonary artery results in more rapid flow and therefore shall result in a shorter lag time.

It should be noted, however, that, if the increase in pressure is due to an increase in pulmonary vascular resistance, that the velocity of blood flow will increase and the pressure will be reduced after encountering the resistance. Therefore, measurement at a point very close to the pulmonary outflow from the heart is necessary, and the optimal place for measuring it is through the airway adjacent the proximal pulmonary artery, prior to any opportunity for any intrinsic vascular flow restriction.

The lag time is the time between a heart contraction, as measured by the EKG or ECG, and in particular, by the R wave of the QRS complex, and the pulmonary pulse (e.g., the periodic expansion of the pulmonary artery). It may be measured using various instruments.

For example, in one variation, the lag time is measured as the difference in time (peak to peak) between a patient's EKG signal, and a pressure pulse signal of the pulmonary artery. The pressure pulse signal may be measured using an ultrasound Doppler catheter positioned in the airway adjacent the pulmonary artery. A bronchial Doppler catheter is adapted to detect velocity profiles through the pulmonary artery. The Doppler catheter can detect the pulsing flow of the blood. The EKG and the velocity signals are measured and charted on the same time scale. The lag time between signal peaks is recorded.

In addition to the Doppler measurement discussed above, additional techniques may measure the lag time including but not limited to a) measuring the strain versus time; video analysis of movement versus time, pressure versus time, and pulse oximetry. In each case, the time delay between the EKG peak and the peak signal using the second minimally invasive modality is recorded.

Below are a number of techniques that provide a signal with which to determine a lag time.

A strain gauge or transducer positioned across a surface of the airway wall shall be subject to periodic movement arising from the pulmonary arterial pulse. The transducer may be carried by a bronchial catheter. The transducer measures the displacement as a function of time. This signal shall follow the EKG signal by a lag time.

Analysis of a set of video frames from an endoscope and in particular, observing the tissue or lumen outlines being displaced as a function of time shall indicate pulse. A computer may store a digital image or picture and record displacement versus time. The peak displacement may be identified and the lag time from the peak of the EKG signal to the peak tissue displacement may thus be calculated.

A bronchial balloon catheter is another instrument that may provide the lag time. The bronchial balloon catheter is positioned in the airway at a location that is subject to the pulsatile flow in the pulmonary artery. The change in pressure as a function of time shall follow the movement of the pulse. The peak displacement may be identified and the lag time from the peak of the EKG signal to the peak displacement thus calculated.

Pulse oximetry is another technique for observing the characteristics of blood flow. A pulse of light is transmitted through tissue and a signal detected on the opposite side, or reflected. The amount of light detected corresponds to the amount of absorption in the spectrum detected if transmitted or the amount reflected by the red cells in the blood. The wavelength of the light, of course, is selected such that it is affected by the red blood cells.

In a variation, a wavelength of light is selected that is affected by deoxygenated blood cells and namely, the blood cells flowing through the pulmonary artery. The blood flowing through the pulmonary artery is substantially deoxygenated. It is thus desirable to select a wavelength of light that shall be affected by the color of the deoxygenated blood cells such as blue or purple, or another wavelength of light. Transmitting such a light through the thorax (from one side of the chest to the other) may thus be affected by the pulsatile motion of the blood cells that are deoxygenated. Though this may not provide an absolute measurement of the flow, concentration, or pressure of the pulmonary artery, it is believed to show a tidal or pulsatile movement when recorded versus time. The peak of the deoxygenated red cell flow may be identified and the lag time from the peak of the EKG signal to the peak of the red cell flow thus calculated.

A pulse oximetry detection device may also be positioned in close proximity to the blood to be detected. A pulse oximetry catheter may be advanced through the airway and near the pulmonary artery. Further, the pulse oximetry catheter may be advanced through the airway, and through a hole in an airway to access the pulmonary artery directly. Similar to the embodiment discussed above, the measurements record a flow or pulse corresponding to the movement of deoxygenated blood cells through the pulmonary artery.

Accordingly, in each of the above cases, the lag time is observed over time and compared to a baseline value. The baseline may be established from a right heart catheterization or another technique to provide an absolute pressure measurement. The baseline value is taken in combination with a baseline lag time. Should the lag time change over time, and in particular, should the lag time decrease from the baseline measurement, pulmonary hypertension may be present. Characteristic lag times may also be identified by studying both healthy and diseased patients using the inventive methods described in this application and comparing those to values received from other techniques, such as right heart catheterization.

A variation of the invention includes taking measurements of the lag time periodically (e.g., monthly, quarterly, or annually). In another variation, the measurements are taken following a treatment or therapy such as a drug therapy, interventional procedure, or conservative treatment. A medicant, such as Viagra®, is injected into or injested by the patient. The measurements are compared and a trend or change between measurements is noted. It is not necessary that the second modality measurement provides an absolute measurement. The second modality is indicative of a pulmonary pressure trend or change from the baseline.

In another variation of the invention, anatomical dimensions of the patient are combined with the lag time measurement to determine flowrate (Q). The dimensions of the pulmonary artery are calculated from, for example, preoperative or live scanning and imaging techniques. In this manner, the length of travel through the pulmonary artery, and the diameter of the pulmonary artery calculated. The length measurement is the distance from the exit of the right ventricle to the location at which the bronchial instrument is positioned (e.g., the location of the bronchial ultrasound Doppler catheter, or the bronchial pressure measurement balloon, the strain gauge, etc.). Additionally, an estimate of flowrate through the pulmonary artery may be computed.

Another variation of the invention includes a method to build a database of patient data from multiple subjects. The database correlates patient lag time, patient anatomical dimensions, and an absolute value of the pulmonary pressure. The data may be presented in various forms such as, for example, a table, computer database, or written chart.

The data is generated by making pressure measurements using a well known technique such as the right heart catheterization. The absolute pressure is recorded along with the subject's dimensions of the pulmonary vessel. In particular, length and diameter are recorded. The database is filled with empirical data of numerous (preferably hundreds of) patients.

FIG. 5 is a hypothetical chart representing a variation of the invention. With reference to the chart shown in FIG. 5, the horizontal axis represents the lag, and the vertical axis represents the absolute pressure. A family of lines is shown, each of which represents a characteristic vessel dimension such as diameter (Φ). In an application or perhaps, a diagnosis, of pulmonary hypertension, the physician measures the lag time along a known length of vessel using one of the techniques described herein. The physician refers to the chart and identifies the vessel diameter (Φ) and selects the corresponding line of the chart from the family of lines shown. The chart correlates the lag time to the pressure based on the previously generated empirical data. Since the line represents the empirical correlation of the vessel diameter, the lag time, and the pressure, the pressure for a new subject may be estimated by measuring only the lag time and the diameter of the vessel. Thus, once the database is complete, pulmonary hypertension may be tracked without the need of a right heart catheterization. Notably, in this variation, the estimated pressure is an absolute measure of pressure.

In another variation, pressure information is measured in the peripheral or radial blood vessels including a radial pressure and a radial pulse lag time. Additionally, a pulmonary artery lag time is measured using one of the above described techniques. The pressure measurement in combination with the lag time measurements should allow for the calculation of the pulmonary arterial pressure. This follows from the principle that the volume of blood coming to the periphery per heart beat is the same as the amount going to the lungs in each heart beat over the same period of time. As a result, the ratio of pressure and resistance, which is equal to flow, is the same in the lungs and in the periphery. Since resistance is the only variable affecting pressure, and it is possible to easily measure the pressure in the periphery with a blood pressure cuff, for instance, and because the lag time is directly related only to the resistance, the lag time from the heartbeat to the pulse in the lung periphery is related to the lag time in the peripheral artery. The ratio of these lag times is equal to the ratio of the peripheral blood pressure to the pulmonary blood pressure. By multiplying the peripheral blood pressure by the ratio of the lag time peripherally divided by the lag time in the pulmonary periphery, you obtain the pulmonary pressure.

The invention has been described with reference to various techniques, methods, and instruments. The invention however may also include a workstation to carry out any of the above techniques. A workstation may include a computer or controller with one or more connections to cooperate with various instruments or sensors (e.g., strain gauge catheter, pressure transducer, thermistor, or EKG). A computer may be adapted to collect and store data, correlate data, compute results (e.g., using formulas such as Hooke's law, continuity equation, and Bernoulli's equation), control sensors (e.g., control expansion of a balloon or injection of a bolus of fluid), or activate an ultrasonic transducer. The invention may comprise the workstation including one or more catheters, a programmed computer or controller, display, software, and/or convenient user interface such as a keyboard, mouse, or another input device.

It is understood that variations of the above methods may include combinations of aspects of each described method as well as combinations of the methods themselves. In addition, the above methods are intended to illustrate the overall benefits of measuring pulmonary hypertension from within the airways. It is understood the teachings herein may be combined with the knowledge of those skilled in the art to yield the necessary measurements or comparisons of measured information.

We claim:

1. A method for observing a pressure in a pulmonary artery, the method comprising:
advancing an expandable device within an airway of a patient;
pressurizing the expandable device against an airway wall to compress a pulmonary artery adjacent to the airway wall in a sufficient amount to interfere with a flow of blood in the pulmonary artery;
observing a pressure of the expandable device; and
correlating the interference of the flow of blood in the pulmonary artery with the pressure in the expandable device to determine the pressure of the pulmonary artery.

2. The method of claim 1 wherein the expandable device is advanced within the airway and to a location in a vicinity of a ligamentum arteriosum

3. The method of claim 1, where pressuring the expandable device comprises stopping the flow of blood in the pulmonary artery.

4. The method of claim 3, where observing the pressure of the expandable device comprises observing the pressure when blood flow stops.

5. The method of claim 4, further comprising subsequently depressurizing the expandable device to re-establish blood flow, where observing the pressure of the expandable device comprises observing the pressure when blood flow is re-established.

6. A method for observing a pressure in a pulmonary artery, the method comprising:
advancing a first device into an airway;
measuring a first flow rate of blood flow in the pulmonary artery with the first device;
pressurizing an expandable member within an airway to interfere with the blood flow in the pulmonary artery;
measuring a second flow rate of blood flow in the pulmonary artery; and
comparing the first flow rate to the second flow rate to determine the pressure in the pulmonary artery.

7. A method for observing a pressure in a pulmonary artery, the method comprising:
locating the pulmonary artery along a surface of the airway;
advancing a device into an airway of a lung; inserting a pressure measuring device through the airway wall directly into the pulmonary artery;
measuring the pressure of the pulmonary artery with the pressure measuring device;
removing the pressure measuring device from the pulmonary artery.

8. The method of claim 1, further comprising sealing an opening in the pulmonary artery created by the advancement of the measuring device.

9. The method of claim 8, wherein sealing is carried out with a tamponade.

10. A method for observing a pressure in a pulmonary artery, the method comprising:
locating the pulmonary artery along a surface of the airway;
placing a motion sensing device against the surface of the airway near a location of the pulmonary artery;
measuring a change in a physical parameter of a wall of the pulmonary artery resulting from an increase in pressure in the pulmonary artery through the airway wall;
determining a radius of the pulmonary artery; and
calculating an increase in pressure of the pulmonary artery using the amount of movement of the airway wall and the radius of the pulmonary artery.

11. The method of claim 10, where the motion sensing device-comprises an accelerometer device.

12. The method of claim 10, where the motion sensing device comprises a strain gauge device

13. A method for observing a pressure in a pulmonary artery, the method comprising:
locating the pulmonary artery along a surface of the airway;
advancing a device into an airway of a lung, where the device is configured to measure a Doppler flow velocity pattern or waveform in the pulmonary artery;
recording waveforms of blood flow with the device over a period of time; and
comparing measured waveforms of blood flow to characteristic waveforms to approximate the pressure in the pulmonary artery.

14. The method of claim 13, where recording waveforms of blood flow over the period of time comprises recording waveforms over several beats of a heart.

15. A method for observing a pressure in a pulmonary artery, the method comprising:
providing a catheter comprising a distal portion configured for placement within the artery and a sensor associated with the distal portion;
advancing the distal portion of the catheter and the sensor to a location within the airway, said location having pulsatile motion arising from the pulmonary artery;
determining said pressure of the pulmonary artery using information obtained from said sensor.

16. The method of claim 15, wherein said sensor is one sensor selected from the group consisting of a thermistor, a strain gauge, an ultrasonic transducer.

17. A method for observing a pressure change in a pulmonary artery of a patient, the method comprising:
measuring an absolute pulmonary baseline pressure and a baseline lag time corresponding to said absolute pulmonary baseline pressure, said baseline lag time being a difference in time between a heart contraction and a subsequent pulse movement in the pulmonary artery;
measuring a real lag time;
comparing the real lag time to said baseline lag time.

18. The method of claim 17, wherein the real lag time is measured with one instrument selected from the group consisting of an ultrasonic Doppler catheter, pulse oximeter, balloon catheter, strain gauge, thermistor, and bronchoscope.

19. The method of claim 18, wherein the real lag time is measured using a balloon catheter in fluid connection with a pressure gauge.

20. A method for observing a pressure in a pulmonary artery through which blood is flowing, the method comprising
advancing a device into an airway within the lung and in the vicinity of the pulmonary artery; and
measuring a characteristic of said blood flowing through said pulmonary artery.

21. The method of claim 20, further comprising determining the pulmonary pressure.

22. The method of claim 20, further comprising advancing a needle into the pulmonary artery, and said needle carrying a pressure transducer into the pulmonary artery.

23. The method of claim 20, wherein measuring is performed by inflating a balloon against the airway wall to the extent that the flow of blood through the pulmonary artery is affected.

24. The method of claim 20, wherein measuring is performed by detecting a signature waveform corresponding to said blood flowing through said pulmonary artery.

25. A noninvasive method for observing a pulmonary artery pressure, the method comprising:
placing an instrument on the chest of a patient, and over the lungs;
sensing a characteristic of the blood flow in the lungs with said instrument where said characteristic is indicative pulmonary blood flow through the lungs.

26. The method of claim 25, wherein said instrument directs light at the lungs, and said light has a wavelength in the range that is affected by deoxygenated blood cells.
